(19) United States
(12) Patent Application Publication
(10) Pub. No.: US 2026/0151447 A1
(43) Pub. Date: Jun. 4, 2026

(54) LONG-ACTING TICK-REPELLING AND KILLING COMPOUNDED PLANT ESSENTIAL OIL NANO-EMULSION AND PREPARATION METHOD THEREOF

(71) Applicants: Henan Agricultural University, Zhengzhou (CN); Henan Hemu Animal Pharmaceutical Co., Ltd., Zhengzhou (CN); Yongcheng Livestock Development Service Center, Shangqiu (CN)

(72) Inventors: Fuchun JIAN, Zhengzhou (CN); Senyang LI, Zhengzhou (CN); Yifei LIU, Zhengzhou (CN); Yichen JIAN, Zhengzhou (CN); Dongliang LI, Zhengzhou (CN); Qing GUO, Zhengzhou (CN); Sumei ZHANG, Zhengzhou (CN); Longxian ZHANG, Zhengzhou (CN); Changshen NING, Zhengzhou (CN); Longfei CHEN, Shangqiu (CN)

(73) Assignees: Henan Agricultural University, Zhengzhou (CN); Henan Hemu Animal Pharmaceutical Co., Ltd., Zhengzhou (CN); Yongcheng Livestock Development Service Center, Shangqiu (CN)

(21) Appl. No.: 19/273,242

(22) Filed: Jul. 18, 2025

(30) Foreign Application Priority Data
Nov. 30, 2024 (CN) 202411743634.6

(51) Int. Cl. A61K 36/79; A61K 9/00; A61K 9/107; A61K 36/185; A61K 47/10; A61K 47/26; A61P 33/14

(52) U.S. Cl. CPC A61K 36/79; A61K 9/0017; A61K 9/1075; A61K 36/185; A61K 47/10; A61K 47/26; A61P 33/14

(57) ABSTRACT

A long-acting tick-repelling and killing compounded plant essential oil nano-emulsion and a preparation method thereof are provided. The nano-emulsion is prepared from materials including the components in weight percentages: 3% of star anise essential oil, 3% of Hydnocarpi Semen essential oil, 21.6-27% of absolute ethanol, 21.6-27% of Tween-80, with the balance being distilled water. Tween-80 (21.6 g) and absolute ethanol (21.6 g) are uniformly mixed to obtain a mixed solution. The long-acting tick-repelling and killing compounded plant essential oil nano-emulsion shows good stability and does not undergo oil-water separation when left for long periods of time. The nano-emulsion provides a long-acting repellent effect against tick attacks, exerts killing effects on ticks attached to the animal's body surface, and causes minimal irritation to animals. It can enhance the bioavailability of active pharmaceutical ingredients, avoids issues of drug resistance and residue, and is eco-friendly.

### CROSS-REFERENCE TO RELATED APPLICATIONS

This application is based upon and claims priority to Chinese Patent Application No. 202411743634.6, filed on Nov. 30, 2024, the entire contents of which are incorporated herein by reference.

### TECHNICAL FIELD

The present disclosure relates to the technical field of traditional Chinese veterinary medicine as alternatives to antibiotics, in particular to a long-acting tick-repelling and killing compounded plant essential oil nano-emulsion and a preparation method thereof.

### BACKGROUND

Ticks are obligate blood-sucking ectozoic parasites that are parasitic on the body surface of animals and are widely distributed around the world. Tick bites can cause host animals to suffer from emaciation, anemia and paralysis and lead to growth retardation in young livestock, while simultaneously transmitting multiple zoonotic pathogens such as Piroplasmida, Anaplasmataceae, Brucella, and Bunya virus. The widespread transmission and infestation of ticks not only poses a significant threat to the health of livestock and poultry but also causes significant economic loss and burden to the global animal husbandry industry and public health security.

Currently, tick control relies heavily on chemical agents, primarily cyfluthrin, fenvalerate, ivermectin, etc. In some regions, farmers often use multiple chemical agents in combination due to severe tick threat, which has led to problems such as drug resistance and persistent environmental pollution. The long-term extensive use and environmental accumulation of these chemical agents cause irreversible damage to the ecological environment, raising the question of whether the current tick control methods are environmentally friendly and sustainable. To achieve scientific tick control that avoids drug resistance, environmental residues, and adverse effects on animals, there is an urgent need to develop an eco-friendly, long-acting alternative product for repelling and killing ticks, which would further alleviate the environmental pollution and drug resistance caused by chemical agents. Therefore, we propose a long-acting tick-repelling and killing compounded plant essential oil nano-emulsion and a preparation method thereof.

### SUMMARY

The object of the present disclosure is to at least address one of the drawbacks existing in the conventional technology and the issues raised in the background section.

In order to achieve the object described above, the present disclosure provides the following technical solutions: a long-acting tick-repelling and killing compounded plant essential oil nano-emulsion and a preparation method thereof. Alternatively, the nano-emulsion (prepared amount 100 g) is prepared from materials including the components in weight percentages: 3% of star anise essential oil, 3% of Hydnocarpi Semen essential oil, 21.6-27% of absolute ethanol, and 21.6-27% of Tween-80 at a cosurfactant-to-surfactant ratio=1:1, and distilled water as balance.

Preferably, Tween-80 (21.6 g) and absolute ethanol (21.6 g) are uniformly mixed to obtain a mixed solution.

Preferably, 3 g of star anise essential oil and 3 g of Hydnocarpi Semen essential oil are added into the mixed solution obtained in the above-mentioned step, the resulting mixture is stirred at a constant speed of 1200 r/min, and 50.8 g of distilled water is slowly added while the mixture is stirred for 30 min to obtain a clear solution.

Preferably, upon completion of the above-mentioned steps, the resulting solution is transferred to a high-speed shearing machine and sheared for 30 min at 26000 r/min to obtain a stable nano-emulsion.

In one embodiment of the present disclosure, the long-acting tick-repelling and killing compounded plant essential oil nano-emulsion is prepared by the following steps:
Step 1: mixing Tween-80 with absolute ethanol a form a first mixture;
Step 2: adding star anise essential oil, Hydnocarpi Semen essential oil, and distilled water into the first mixture to form a second mixture;
Step 3: mixing the second mixture in a high-speed shearing machine; and
Step 4: allowing second mixture to stand to afford the long-acting tick-repelling and killing compounded plant essential oil nano-emulsion.

Compared with conventional technologies, the beneficial effects of the present disclosure are as follows:

(1) The long-acting tick-repelling and killing compounded plant essential oil nano-emulsion is in the form of uniformly distributed, suspended particles with a small size of 20-80 nm and shows good stability and no oil-water separation occurs when left for a long period of time. When in use, the nano-emulsion can be directly sprayed onto the body surface of livestock and pets. It provides a long-acting repellent effect against tick attacks, exerts killing effects on ticks attached to the animal's body surface, and causes minimal irritation to animals. It can enhance the bioavailability of active pharmaceutical ingredients, avoids issues of drug resistance and residue, and is eco-friendly.

(2) Compared to traditional tick repellents, the long-acting tick-repelling and killing compounded plant essential oil nano-emulsion utilizes the oil-in-water preparation technology, which not only ensures rapid efficacy, but also effectively improves drug utilization, storage stability, and pesticide duration. It also reduces production costs, safeguards ecological safety, and integrates economic, social, and environmental benefits. This is why the nano-emulsion is an optimal choice for today's antibiotic-free pest control.

(3) In the long-acting tick-repelling and killing compounded plant essential oil nano-emulsion, plant essential oils are fully encapsulated by surfactants, resulting in a safe, stable, and effective plant essential oil nano-emulsion. The final product can exert both toxic and repellent effects on ticks and can effectively prolong the pesticide duration of the plant essential oil nano-emulsion.

(4) The long-acting tick-repelling and killing compounded plant essential oil nano-emulsion features a water molecular layer coated on the drug surface, preventing direct environmental exposure. This design eliminates environmental pollution and ensures no toxic effects on non-target organisms.

(5) In the long-acting tick-repelling and killing compounded plant essential oil nano-emulsion and preparation method thereof, plant essential oils can be fully encapsulated by surfactants, resulting in a safe, stable, and effective plant essential oil nano-emulsion. The final product can exert both toxic and repellent effects on ticks and can effectively prolong the pesticide duration of the plant essential oil nano-emulsion. The preparation process significantly shortens production time and improves production efficiency without compromising the physical properties or quality of the formulations.

### BRIEF DESCRIPTION OF THE DRAWINGS

The present disclosure will be further described below with reference to the accompanying drawings and embodiments:

FIG. 1 is a schematic diagram of the post-administration repellency rate variation curve in the present disclosure; and

FIG. 2 shows the data on the therapeutic efficacy of essential oils on high-density tick infected goats.

### DETAILED DESCRIPTION OF THE EMBODIMENTS

Specific embodiments of the present disclosure will be described in detail in this section. Preferred embodiments of the present disclosure are illustrated in the accompanying drawings. The accompanying drawings serve to supplement the text description of the description with figures, providing a visual understanding of each technical feature and the overall technical solution of the present disclosure, but cannot be construed as a limitation to the scope of protection of the present disclosure.

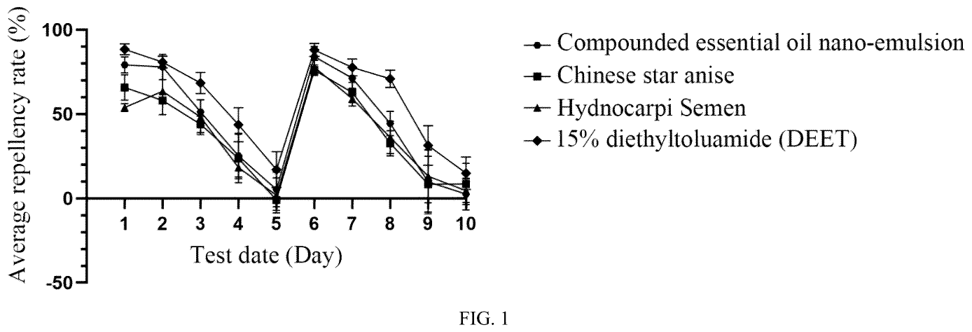

Referring to FIG. 1, the present disclosure provides a long-acting tick-repelling and killing compounded plant essential oil nano-emulsion and a preparation method thereof. Based on needs in first line breeding, the inventors conducted screening tests and efficacy evaluation on various insect-resistant plant extracts, selected effective drugs for the formulation, and performed formulation research to obtain a compounded essential oil micro-emulsion preparation with ideal efficacy, specifically as follows:

### Example 1

The nano-emulsion of this example consists of the following components in weight percentages: 3% of star anise essential oil, 3% of Hydnocarpi Semen essential oil, 21.6% of absolute ethanol, 21.6% of Tween-80, and 50.2% of distilled water.

The preparation method of the long-acting tick-repelling and killing compounded plant essential oil nano-emulsion includes the steps of:
(1) uniformly mixing Tween-80 and absolute ethanol to obtain a mixture;
(2) simultaneously adding star anise essential oil and Hydnocarpi Semen essential oil to the mixture obtained in step (1), uniformly mixing the resulting mixture, adding distilled water under stirring, stirring the resulting mixture until a homogeneous and clear system is formed, and then stirring the mixture at 1200 r/min for 12 h to obtain the target product.

Further, after the aforementioned steps and operations, the plant essential oils can be fully encapsulated by surfactants, thereby resulting in a safe, stable, and effective plant essential oil nano-emulsion. The product obtained in this step can exert both toxic and repellent effects on ticks and can effectively prolong the pesticide duration.

### Example 2

The nano-emulsion of this example includes the following components in weight percentages: 3% of star anise essential oil, 3% of Hydnocarpi Semen essential oil, 21.6% of absolute ethanol, 21.6% of Tween-80, and 50.2% of distilled water.

The preparation method of the long-acting tick-repelling and killing compounded plant essential oil nano-emulsion includes the steps of:
(1) uniformly mixing Tween-80 and absolute ethanol to obtain a mixture;
(2) simultaneously adding star anise essential oil and Hydnocarpi Semen essential oil to the mixture obtained in step (1), uniformly mixing the resulting mixture, adding distilled water under stirring, stirring the resulting mixture until a homogeneous and clear system is formed, and then stirring the mixture at 1200 r/min for 30 min to form a clear liquid;
(3) transferring the clear liquid obtained in step (2) to a high-speed shearing machine and stirring the clear liquid at 26000 r/min for 30 min to obtain the final product.

Further, after the aforementioned steps and operations, the plant essential oils can be fully encapsulated by surfactants, thereby resulting in a safe, stable, and effective plant essential oil nano-emulsion. The product obtained in this step can exert both toxic and repellent effects on ticks and can effectively prolong the pesticide duration. The preparation process significantly shortens production time and improves production efficiency without compromising the physical properties or quality of the preparation.

Still further, efficacy determination tests were conducted.

Test Example 1 for Efficacy Determination (Repellency Test): In this application example of the preparation, the repellent activity of the compounded essential oil nano-emulsion obtained in Example 2 was determined against ticks. The test method was as follows:
Test subjects: 25 free-ranging goats naturally infected with ticks were allocated into five groups, with five animals in each group;
Blank control (water) group: 30 to 40 mL of tap water was sprayed onto each animal;
Star anise essential oil micro-emulsion group: 30 to 40 mL of the drug was sprayed onto each animal;
Hydnocarpi Semen essential oil micro-emulsion group: 30 to 40 mL of the drug was sprayed onto each animal;
Compounded essential oil nano-emulsion group: 30 to 40 mL of the drug was sprayed onto each animal;
Positive control group (15% diethyltoluamide): 30 to 40 mL of the drug was sprayed onto each animal;
Administration: drug infiltration was allowed. The administration sites were rubbed after drug administration to prolong the drug adhesion time;
Administration frequency: one dose every five days, two doses in total;
Grazing: before the experiment, ticks were removed from the experimental goats. After grouping and labeling, all flocks of experimental goats were randomized and mixed, then grazed in the same flock and at the same location. After grazing, the number of hard ticks on the goats was recorded daily and compared with the control group, and the repellency rate was calculated. After recording, the hard ticks were removed from the goats, and the experiment proceeded to the next day. The average repellency rate was calculated based on the obtained data;
Counting sites: limbs, armpits, ears, neck, and face;

repellency rate (PR)=(1−number of hard ticks in the treatment group/number of hard ticks in the control group)×100%;

Test results: it can be seen from FIG. 1 that the compounded essential oil micro-emulsion exhibits tick control efficacy comparable to that of the positive control drug (insignificant difference), effectively repelling ticks and thereby protecting individual animals.

Furthermore, compared to the experimental positive control drug, this product of the present disclosure offers the advantages as follows: (1) effectively reduced drug cost: the price of the experimental positive control drug is five times that of the compounded plant nano-essential oil; (2) low toxicity: it has no the pungent odor associated with the positive control drug and has no toxic effects on the animals; (3) no drug residue: due to the special characteristics of traditional Chinese medicine products, which have multiple ingredients and targets, there is no drug residue after use; this avoids inducing drug resistance in ticks, extends the effective usage period of the drug, and ensures biosafety; (4) environmental friendliness: when released into the environment, it produces no toxic and side effects on non-target organisms and flora/fauna, preserving environmental biodiversity.

Test Example 2 for Efficacy Determination (Treatment Test): In this test example, the repelling and killing activity of the compounded essential oil nano-emulsion obtained in Example 2 was determined against ticks. The test method was as follows:
The animals were divided into 5 groups; (1) blank (water) control group (3 animals),
(2) Positive (15% diethyltoluamide) control group (3 animals),
(3) star anise essential oil micro-emulsion group (3 animals),
(4) Hydnocarpi Semen essential oil micro-emulsion group (3 animals),
(5) Compounded essential oil micro-emulsion group (3 animals).

In this test, goats that were heavily infested with ticks in the ear region were selected for the treatment test. Before the test, ticks in the ear region were counted, and drug administration was followed. After 24 hours, the ticks in the ear region were counted again. The drugs were administered twice in a row in a dose of 5-7 mL each time. The tick reduction rate in the ear region of the test goats was calculated to evaluate the treatment effects.

Treatment Efficacy=(1−Number of post-administration hard ticks/Number of pre-administration hard ticks)×100%. Reference may be made specifically to Table 1.

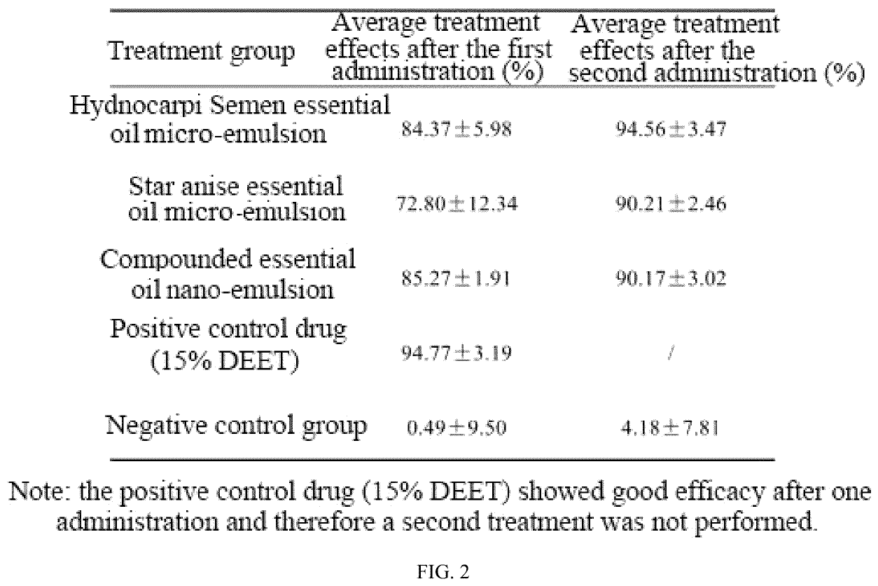

It can be seen from FIG. 2 that when free-ranging goats were heavily infested with ticks, spraying the compounded essential oil micro-emulsion can effectively produce tick-repelling and killing effects, achieving a tick repellency rate of at least 90%.

Further, compared to traditional tick repellents, the product of the present disclosure utilizes the oil-in-water preparation technology, which not only ensures rapid efficacy, but also effectively improves drug utilization, storage stability, and pesticide duration. It also reduces production costs, safeguards ecological safety, and integrates economic, social, and environmental benefits. This is why the nano-emulsion is an optimal choice for today's antibiotic-free pest control.

The embodiments of the present disclosure are described in detail above with reference to the accompanying drawings, but the present disclosure is not limited to the foregoing embodiments, and various changes can be made without departing from the objectives of the present disclosure within the scope of knowledge possessed by those of ordinary skill in the art.

What is claimed is:

1. A method for preparing a long-acting tick-repelling and killing compounded plant essential oil nano-emulsion, wherein the long-acting tick-repelling and killing compounded plant essential oil nano-emulsion comprises the following components in weight percentages: 3% of star anise essential oil, 3% of Hydnocarpi Semen essential oil, 21.6-27% of absolute ethanol, and 21.6-27% of Tween-80 at a cosurfactant-to-surfactant ratio of 1:1, and distilled water as a balance;
wherein the method comprises the following steps:
1) selecting materials for the long-acting tick-repelling and killing compounded plant essential oil nano-emulsion for later use;
2) mixing and stirring the materials to obtain a first mixture;
3) subjecting ingredients of the materials to a second loading, mixing and stirring to obtain liquid materials;
4) charging all the liquid materials into a high-speed shearing machine for a further stirring to obtain a second mixture; and
5) leaving the second mixture to stand after stirring to obtain the long-acting tick-repelling and killing compounded plant essential oil nano-emulsion.

2. The method according to claim 1, wherein the materials for the compounded plant essential oil nano-emulsion compounded plant essential oil nano-emulsion in step 1) comprise the components in weight percentages: 3% of star anise essential oil, 3% of Hydnocarpi Semen essential oil, 21.6-27% of cosurfactant and 21.6-27% of surfactant at the cosurfactant-to-surfactant ratio of 1:1, and distilled water as balance.

3. The method according to claim 2, wherein based on step 2), 21.6% of surfactant and 21.6% of cosurfactant are uniformly mixed.

4. The method according to claim 3, wherein in step 3), 3% of star anise essential oil and 3% of Hydnocarpi Semen essential oil are simultaneously added into the materials, the mixing under stirring is performed at a constant speed of 1200 r/min in a stirring device, and then 50.8% of distilled water is added under stirring.

5. The method according to claim 4, wherein in steps 4) and 5), the liquid materials are added to the high-speed shearing machine to obtain a homogeneous and stable nano-emulsion.

6. The method according to claim 3, wherein the surfactant is Tween-80, and the cosurfactant is absolute ethanol.

7. The method according to claim 5, wherein the long-acting tick-repelling and killing compounded plant essential oil nano-emulsion obtained in step 5) is in a form of uniformly distributed and suspended particles with a small particle size of 20-80 nm, and shows a good stability, and does not undergoes oil-water separation when left for long periods of time.

8. The method according to claim 4, wherein after the star anise essential oil and the Hydnocarpi Semen essential oil are simultaneously added into the first mixture, the resulting mixture is mixed for 30 min-12 h.

9. The method according to claim 5, wherein the high-speed shearing machine is operated at a speed of 26000 r/min for 30 min.

10. The method according to claim 4, wherein the star anise essential oil is originated from star anise, and the Hydnocarpi Semen essential oil is originated from traditional Chinese medicine Hydnocarpi Semen.

11. A long-acting tick-repelling and killing compounded plant essential oil nano-emulsion, comprising components in weight percentages: 3% of star anise essential oil, 3% of Hydnocarpi Semen essential oil, 21.6-27% of absolute ethanol and 21.6-27% of Tween-80 at a cosurfactant-to-surfactant ratio of 1:1, and distilled water as a balance.

12. The long-acting tick-repelling and killing compounded plant essential oil nano-emulsion according to claim 11, wherein the surfactant has a weight percentage of 21.6% and the cosurfactant has the weight percentage of 21.6%.

13. The long-acting tick-repelling and killing compounded plant essential oil nano-emulsion according to claim 11, wherein suspended particles in the long-acting tick-repelling and killing compounded plant essential oil nano-emulsion has a particle size of 20-80 nm.

14. The long-acting tick-repelling and killing compounded plant essential oil nano-emulsion according to claim 12, wherein the surfactant is Tween-80, and the cosurfactant is absolute ethanol.

15. The long-acting tick-repelling and killing compounded plant essential oil nano-emulsion according to claim 11, wherein the star anise essential oil is originated from star anise, the star anise is a medicine and food homology and is non-toxic and harmless, and the Hydnocarpi Semen essential oil is originated from traditional Chinese medicine Hydnocarpi Semen.
